(19) United States
(12) Patent Application Publication
(10) Pub. No.: US 2013/0030431 A1
(43) Pub. Date: Jan. 31, 2013

(54) SHOCK WAVE BALLOON CATHETER SYSTEM WITH OFF CENTER SHOCK WAVE GENERATOR

(71) Applicant: John M. ADAMS, Snohomish, WA (US)

(72) Inventor: John M. ADAMS, Snohomish, WA (US)

(21) Appl. No.: 13/646,583

(22) Filed: Oct. 5, 2012

Related U.S. Application Data
(63) Continuation-in-part of application No. 12/482,995, filed on Jun. 11, 2009.
(60) Provisional application No. 61/061,170, filed on Jun. 13, 2008.

(51) Int. Cl. A61B 18/18; A61B 17/22

(52) U.S. Cl. 606/41; 606/128

(57) ABSTRACT

A catheter comprises an elongated carrier and a balloon about the carrier in sealed relation thereto. The balloon is arranged to receive a fluid therein that inflates the balloon and has an inner surface. The catheter further includes a shock wave generator asymmetrically located within the balloon with respect to the inner surface of the balloon that forms a mechanical shock wave within the balloon. Because the shock wave generator is asymmetrically located within the balloon with respect to the inner surface of the balloon, each shock wave will impact the inner surface of the balloon in a non-uniform manner to prevent the hoop stress of the balloon from being exceeded.

### CROSS-REFERENCE TO RELATED APPLICATION

This application is a continuation-in-part of U.S. application Ser. No. 12/482,995 filed on Jun. 11, 2009 (pending), which application claims the benefit of priority to U.S. Provisional Application No. 61/061,170 filed on Jun. 13, 2008, all of which applications are incorporated herein by reference in their entireties for all purposes.

### BACKGROUND OF THE INVENTION

The present invention relates to a treatment system for percutaneous coronary angioplasty or peripheral angioplasty in which a dilation catheter is used to cross a lesion in order to dilate the lesion and restore normal blood flow in the artery. It is particularly useful when the lesion is a calcified lesion in the wall of the artery. Calcified lesions require high pressures (sometimes as high as 10-15 or even 30 atmospheres) to break the calcified plaque and push it back into the vessel wall. With such pressures comes trauma to the vessel wall which can contribute to vessel rebound, dissection, thrombus formation, and a high level of restenosis. Non-concentric calcified lesions can result in undue stress to the free wall of the vessel when exposed to high pressures. An angioplasty balloon when inflated to high pressures can have a specific maximum diameter to which it will expand but the opening in the vessel under a concentric lesion will typically be much smaller. As the pressure is increased to open the passage way for blood the balloon will be confined to the size of the opening in the calcified lesion (before it is broken open). As the pressure builds a tremendous amount of energy is stored in the balloon until the calcified lesion breaks or cracks. That energy is then released and results in the rapid expansion of the balloon to its maximum dimension and may stress and injure the vessel walls.

### SUMMARY OF THE INVENTION

In one embodiment, a catheter comprises an elongated carrier and a balloon about the carrier in sealed relation thereto. The balloon is arranged to receive a fluid therein that inflates the balloon and has an inner surface. The catheter further includes a shock wave generator asymmetrically located within the balloon with respect to the inner surface of the balloon that forms a mechanical shock wave within the balloon.

The shock wave generator may be an arc generator. The arc generator may include at least one electrode that is asymmetrically located within the balloon. Alternatively, the arc generator may include a pair of electrodes, each electrode of the pair of electrodes being asymmetrically located within the balloon.

In another embodiment, an angioplasty catheter comprises an elongated carrier. The carrier defines a guide wire sheath having a guide wire lumen. The catheter further includes a balloon about the carrier in sealed relation thereto. The balloon has an outer wall, is arranged to receive a fluid therein that inflates the balloon, and has a symmetrical configuration with a center line. The guide wire sheath is centered along the center line of the balloon. The cather further includes an arc generator within the balloon between the guide wire sheath and the balloon outer wall that forms a mechanical shock wave within the balloon.

The arc generator may include at least one electrode located within the balloon between the guide wire sheath and the outer wall of the balloon. Alternatively, the arc generator may include a pair of electrodes, each electrode of the pair of electrodes being located within the balloon between the guide wire sheath and the outer wall of the balloon.

In a still further embodiment, a method comprises the steps of providing a catheter having an elongated carrier, a balloon about the carrier in sealed relation thereto and being arranged to receive a fluid therein that inflates the balloon and a shock wave generator within the balloon. The method further includes inflating the balloon, and causing the shock wave generator to form mechanical shock waves within the balloon from a point asymmetric within the balloon.

The shock wave generator may be an arc generator, and the causing step may include providing the arc generator with voltage pulses.

The catheter may further include a guide wire sheath having a guide wire lumen and the method may further include guiding the catheter along a guide wire received within the guide wire lumen to a desired position before inflating the balloon.

### BRIEF DESCRIPTION OF THE DRAWINGS

For illustration and not limitation, some of the features of the present invention are set forth in the appended claims. The various embodiments of the invention, together with representative features and advantages thereof, may best be understood by making reference to the following description taken in conjunction with the accompanying drawings, in the several figures of which like reference numerals identify identical elements, and wherein:

FIG. 1 is a view of the therapeutic end of a typical prior art over-the-wire angioplasty balloon catheter.

FIG. 2 is a side view of a dilating angioplasty balloon catheter with two electrodes within the balloon attached to a source of high voltage pulses according to one embodiment of the invention.

FIG. 3 is a schematic of a high voltage pulse generator.

FIG. 3A shows voltage pulses that may be obtained with the generator of FIG. 3.

FIG. 4 is a side view of the catheter of FIG. 2 showing an arc between the electrodes and simulations of the shock wave flow.

FIG. 5 is a side view of a dilating catheter with insulated electrodes within the balloon and displaced along the length of the balloon according to another embodiment of the invention.

FIG. 6 is a side view of a dilating catheter with insulated electrodes within the balloon displaced with a single pole in the balloon and a second being the ionic fluid inside the balloon according to a further embodiment of the invention.

FIG. 7 is a side view of a dilating catheter with insulated electrodes within the balloon and studs to reach the calcification according to a still further embodiment of the invention.

FIG. 8 is a side view of a dilating catheter with insulated electrodes within the balloon with raised ribs on the balloon according to still another embodiment of the invention.

FIG. 8A is a front view of the catheter of FIG. 8.

FIG. 9 is a side view of a dilating catheter with insulated electrodes within the balloon and a sensor to detect reflected signals according to a further embodiment of the invention.

FIG. 10 is a pressure volume curve of a prior art balloon breaking a calcified lesion.

FIG. 10A is a sectional view of a balloon expanding freely within a vessel.

FIG. 10B is a sectional view of a balloon constrained to the point of breaking in a vessel.

FIG. 10C is a sectional view of a balloon after breaking within the vessel.

FIG. 11 is a pressure volume curve showing the various stages in the breaking of a calcified lesion with shock waves according to an embodiment of the invention.

FIG. 11A is a sectional view showing a compliant balloon within a vessel.

FIG. 11B is a sectional view showing pulverized calcification on a vessel wall.

FIG. 12 illustrates shock waves delivered through the balloon wall and endothelium to a calcified lesion.

FIG. 13 shows calcified plaque pulverized and smooth a endothelium restored by the expanded balloon after pulverization.

FIG. 14 is a schematic of a circuit that uses a surface EKG to synchronize the shock wave to the “R” wave for treating vessels near the heart.

FIG. 15 is a side view, partly cut away, of a dilating catheter with a parabolic reflector acting as one electrode and provides a focused shock wave inside a fluid filled compliant balloon.

### DETAILED DESCRIPTION OF THE INVENTION

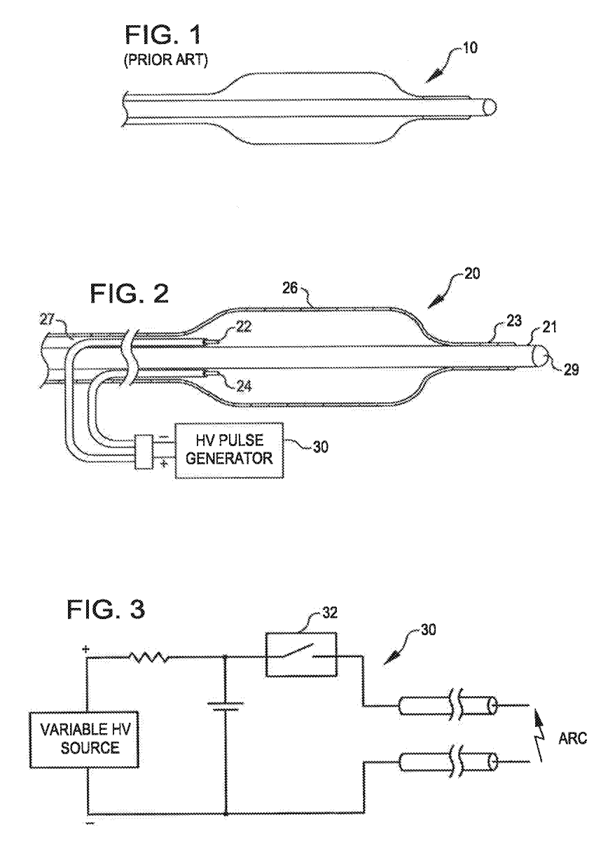

FIG. 1 is a view of the therapeutic end of a typical prior art over-the-wire angioplasty balloon catheter 10. Such catheters are usually non-complaint with a fixed maximum dimension when expanded with a fluid such as saline.

FIG. 2 is a view of a dilating angioplasty balloon catheter 20 according to an embodiment of the invention. The catheter 20 includes an elongated carrier, such as a hollow sheath 21, and a dilating balloon 26 formed about the sheath 21 in sealed relation thereto at a seal 23. The balloon 26 forms an annular channel 27 about the sheath 21 through which fluid, such as saline, may be admitted into the balloon to inflate the balloon. The channel 27 further permits the balloon 26 to be provided with two electrodes 22 and 24 within the fluid filled balloon 26. The electrodes 22 and 24 are attached to a source of high voltage pulses 30. The electrodes 22 and 24 are formed of metal, such as stainless steel or tungsten, and are placed a controlled distance apart to allow a reproducible arc for a given voltage and current. The electrical arcs between electrodes 22 and 24 in the fluid are used to generate shock waves in the fluid. The variable high voltage pulse generator 30 is used to deliver a stream of pulses to the electrodes 22 and 24 to create a stream of shock waves within the balloon 26 and within the artery being treated (not shown). The magnitude of the shock waves can be controlled by controlling the magnitude of the pulsed voltage, the current, the duration and repetition rate. The insulating nature of the balloon 26 protects the patient from electrical shocks.

The balloon 26 may be filled with water or saline in order to gently fix the balloon in the walls of the artery in the direct proximity with the calcified lesion. The fluid may also contain an x-ray contrast to permit fluoroscopic viewing of the catheter during use. The carrier 21 includes a lumen 29 through which a guidewire (not shown) may be inserted to guide the catheter into position. Once positioned the physician or operator can start with low energy shock waves and increase the energy as needed to crack the calcified plaque. Such shockwaves will be conducted through the fluid, through the balloon, through the blood and vessel wall to the calcified lesion where the energy will break the hardened plaque without the application of excessive pressure by the balloon on the walls of the artery.

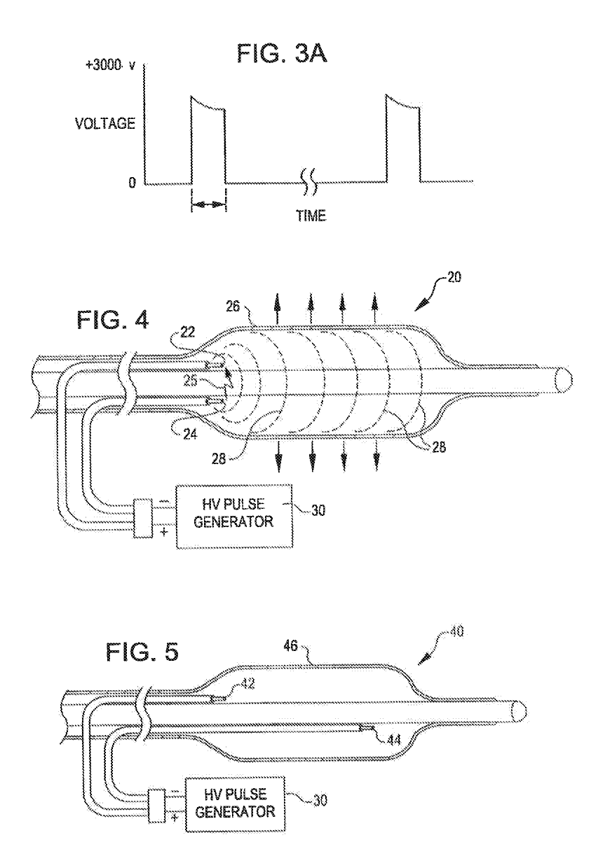

FIG. 3 is a schematic of the high voltage pulse generator 30. FIG. 3A shows a resulting waveform. The voltage needed will depend on the gap between the electrodes and is generally 100 to 3000 volts. The high voltage switch 32 can be set to control the duration of the pulse. The pulse duration will depend on the surface area of the electrodes 22 and 24 and needs to be sufficient to generate a gas bubble at the surface of the electrode causing a plasma arc of electric current to jump the bubble and create a rapidly expanding and collapsing bubble, which creates the mechanical shock wave in the balloon. Such shock waves can be as short as a few microseconds. Since both the rapid expansion and the collapse create shockwaves, the pulse duration can be adjusted to favor one over the other. A large steam bubble will generate a stronger shockwave than a small one. However, more power is needed in the system to generate this large steam bubble. Traditional lithotripters try to generate a large steam bubble to maximize the collapsing bubble's shockwave. Within a balloon such large steam bubbles are less desirable due to the risk of balloon rupture. By adjusting the pulse width to a narrow pulse less than two microseconds or even less than one microsecond a rapidly expanding steam bubble and shockwave can be generated while at the same time the final size of the steam bubble can be minimized. The short pulse width also reduces the amount of heat in the balloon to improve tissue safety.

FIG. 4 is a cross sectional view of the shockwave catheter 20 showing an arc 25 between the electrodes 22 and 24 and simulations of the shock wave flow 28. The shock wave 28 will radiate out from the electrodes 22 and 24 in all directions and will travel through the balloon 26 to the vessel where it will break the calcified lesion into smaller pieces.

FIG. 5 shows another dilating catheter 40. It has insulated electrodes 42 and 44 within the balloon 46 displaced along the length of the balloon 46.

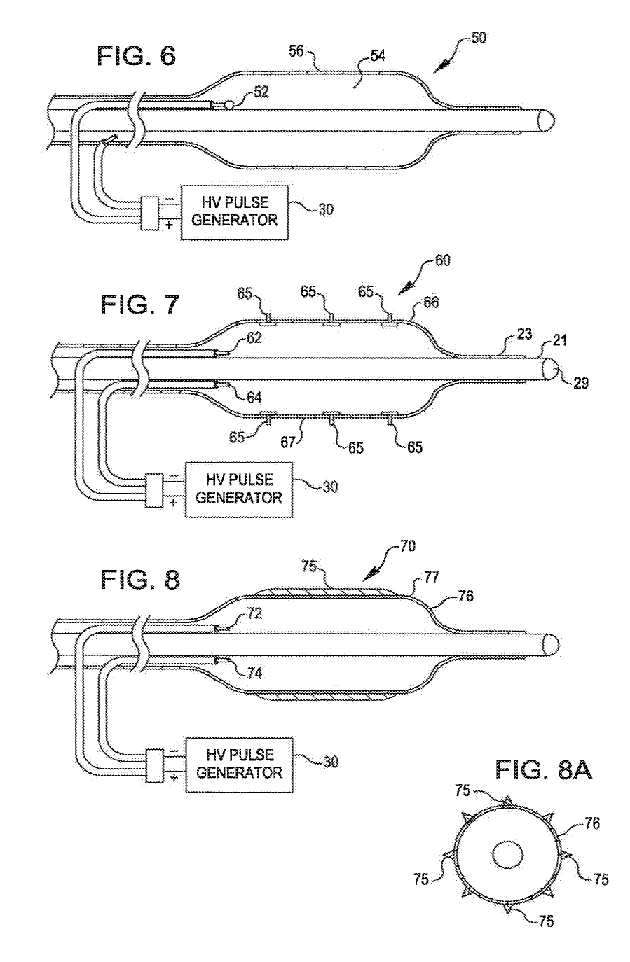

FIG. 6 shows a dilating catheter 50 with an insulated electrode 52 within the balloon 56. The electrode is a single electrode pole in the balloon, a second pole being the ionic fluid 54 inside the balloon. This unipolar configuration uses the ionic fluid as the other electrical pole and permits a smaller balloon and catheter design for low profile balloons. The ionic fluid is connected electrically to the HV pulse generator 30.

FIG. 7 is another dilating 60 catheter with electrodes 62 and 64 within the balloon 66 and studs 65 to reach the calcification. The studs 65 form mechanical stress risers on the balloon surface 67 and are designed to mechanically conduct the shock wave through the intimal layer of tissue of the vessel and deliver it directly to the calcified lesion.

FIG. 8 is another dilating catheter 70 with electrodes 72 and 74 within the balloon 76 and with raised ribs 75 on the surface 77 of the balloon 76. The raised ribs 75 (best seen in FIG. 8A) form stress risers that will focus the shockwave energy to linear regions of the calcified plaque.

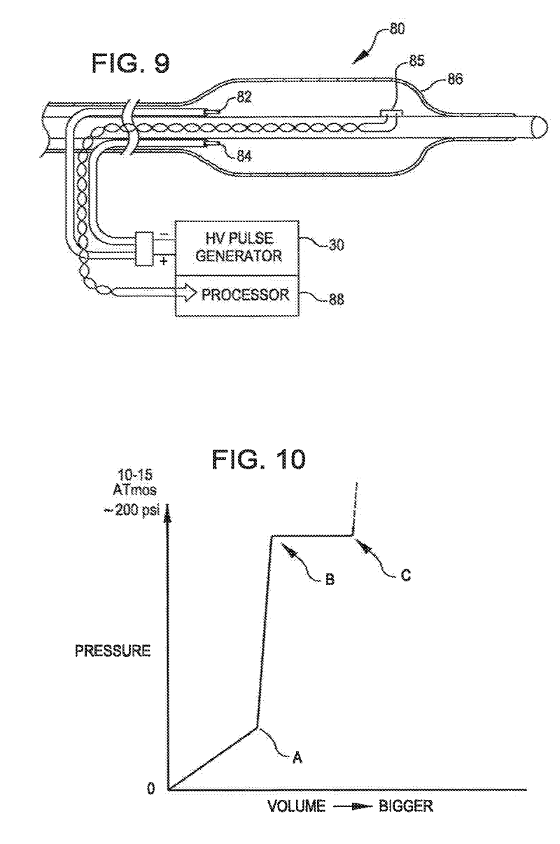

FIG. 9 is a further dilating catheter 80 with electrodes 82 and 84 within the balloon 86.The catheter 80 further includes a sensor 85 to detect reflected signals. Reflected signals from the calcified plaque can be processed by a processor 88 to determine quality of the calcification and quality of pulverization of the lesion.

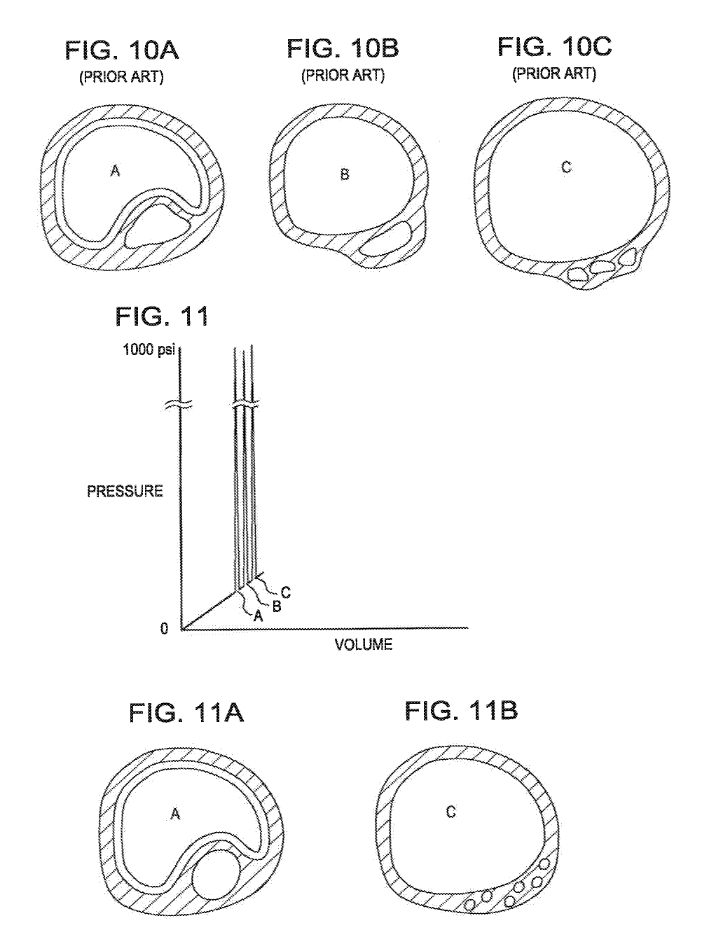

FIG. 10 is a pressure volume curve of a prior art balloon breaking a calcified lesion. FIG. 10B shows the build up of energy within the balloon (region A to B) and FIG. 10C shows the release of the energy (region B to C) when the calcification breaks. At region C the artery is expanded to the maximum dimension of the balloon. Such a dimension can lead to injury to the vessel walls. FIG. 10A shows the initial inflation of the balloon.

FIG. 11 is a pressure volume curve showing the various stages in the breaking of a calcified lesion with shock waves according to the embodiment. The balloon is expanded with a saline fluid and can be expanded to fit snugly to the vessel wall (Region A) (FIG. 11A) but this is not a requirement. As the High Voltage pulses generate shock waves (Region B and C) extremely high pressures, extremely short in duration will chip away the calcified lesion slowly and controllably expanding the opening in the vessel to allow blood to flow un-obstructed (FIG. 11B).

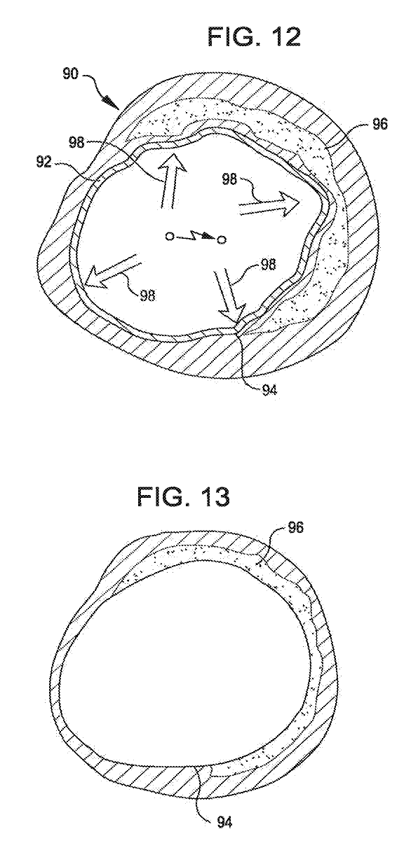

FIG. 12 shows, in a cutaway view, shock waves 98 delivered in all directions through the wall 92 of a saline filled balloon 90 and intima 94 to a calcified lesion 96. The shock waves 98 pulverize the lesion 96. The balloon wall 92 may be formed of non-compliant or compliant material to contact the intima 94.

FIG. 13 shows calcified plaque 96 pulverized by the shock waves. The intima 94 is smoothed and restored after the expanded balloon (not shown) has pulverized and reshaped the plaque into the vessel wall.

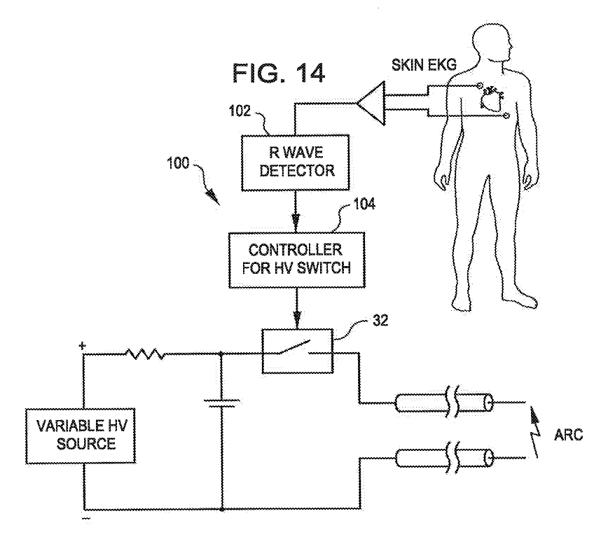

FIG. 14 is a schematic of a circuit 100 that uses the generator circuit 30 of FIG. 3 and a surface EKG 102 to synchronize the shock wave to the “R” wave for treating vessels near the heart. The circuit 100 includes an R-wave detector 102 and a controller 104 to control the high voltage switch 32. Mechanical shocks can stimulate heart muscle and could lead to an arrhythmia. While it is unlikely that shockwaves of such short duration as contemplated herein would stimulate the heart, by synchronizing the pulses (or bursts of pulses) with the R-wave, an additional degree of safety is provided when used on vessels of the heart or near the heart. While the balloon in the current drawings will provide an electrical isolation of the patient from the current, a device could be made in a non-balloon or non-isolated manner using blood as the fluid. In such a device, synchronization to the R-wave would significantly improve the safety against unwanted arrhythmias.

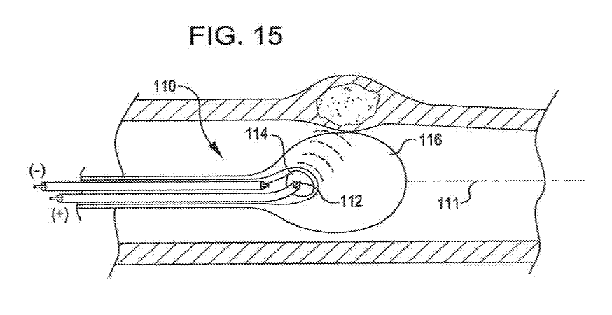

FIG. 15 shows a still further dilation catheter 110 wherein a shock wave is focused with a parabolic reflector 114 acting as one electrode inside a fluid filled compliant balloon 116. The other electrode 112 is located at the coaxial center of the reflector 114. By using the reflector as one electrode, the shock wave can be focused and therefore pointed at an angle (45 degrees, for example) off the center line 111 of the catheter artery. In this configuration, the other electrode 112 will be designed to be at the coaxial center of the reflector and designed to arc to the reflector 114 through the fluid. The catheter or electrode and reflector can be rotated if needed to break hard plaque as it rotates and delivers shockwaves.

Reference is now made again to FIGS. 2,4,5,6, 10 and 11. A typical 4 mm diameter balloon has a 0.6 mm diameter guide wire lumen, such as lumen 29 of FIG. 2. The electrodes 22 and 24 are typically 0.5 mm diameter at the insulation with a wire diameter of 0.25 mm. Thus, in a 4 mm diameter balloon, each of electrodes 22 and 24 is centered about 0.55 mm off the center of the balloon, about 1.45 mm from the closest balloon wall and about 2.55 from the furthest inner balloon wall. The shockwave generated at electrode 22 or electrode 24 will travel at a speed of about 1.5 mm/microsecond. As indicated in FIG. 4 with propagation lines 28, the shockwaves propagate to the balloon walls and down the length of balloon. Thus a shockwave originating from electrode 22 of FIG. 2, electrode 42 of FIG. 5 or electrode 52 of FIG. 6, for example, will hit the closest balloon wall 1.45/1.5=0.97 microseconds after it originates. However, it will hit the furthest balloon wall in the same plane of the electrode 2.55/1.5=1.7 microseconds after its origination. This difference in time is very important given the strength of the shockwaves being on the order of 1000 psi, as may be seen in FIG. 11, and the duration of the shock waves being extremely short, on the order of 1 microsecond or less. The hoop strength of typical angioplasty balloons is on the order of 10 to 20 ATMs (150 to 300 psi) so a pressure pulse of 1000 psi would break the balloon if it were applied uniformly to the opposite sides of the balloon at the same time. A shock wave originating from an electrode centered in a symmetrical balloon would hit the opposed walls of the balloon hoop in the plane of the electrode at the same instant in time and the force thereof would far exceed the hoop strength of the balloon. By placing the electrodes off of the center line of the symmetrical balloon, the hoop strength is not exceeded in any one place or at any instant in time, thus sparing the balloon from rupture. Having the shock source off the center line of the balloon, preferably about 1 mm closer to one wall than the other, protects the balloon from being exposed to hoop stress beyond its limits.

Hence, as may be seen from the above, originating the shock waves asymmetrically within the balloon causes the shock waves to non-uniformly impinge upon the balloon sidewalls. This may be accomplished by locating the shock wave generator non-symmetrically within a symmetrical balloon or by employing a non-symmetrical balloon.

While particular embodiments of the present invention have been shown and described, modifications may be made. It is therefore intended in the appended claims to cover all such changes and modifications which fall within the true spirit and scope of the invention as defined by those claims.

What is claimed is:

1. A catheter comprising:
an elongated carrier;
a balloon about the carrier in sealed relation thereto, the balloon being arranged to receive a fluid therein that inflates the balloon and having an inner surface; and
a shock wave generator asymmetrically located within the balloon with respect to the inner surface of the balloon that forms a mechanical shock wave within the balloon.

2. The catheter of claim 1, wherein the shock wave generator is an arc generator.

3. The catheter of claim 2, wherein the arc generator includes at least one electrode that is asymmetrically located within the balloon.

4. The catheter of claim 2, wherein the arc generator includes a pair of electrodes, each electrode of the pair of electrodes being asymmetrically located within the balloon.

5. An angioplasty catheter comprising:
an elongated carrier, the carrier defining a guide wire sheath having a guide wire lumen;
a balloon about the carrier in sealed relation thereto, the balloon having an outer wall, being arranged to receive a fluid therein that inflates the balloon, and having a symmetrical configuration with a center line, the guide wire sheath being centered along the center line of the balloon; and
an arc generator within the balloon between the guide wire sheath and the balloon outer wall that forms a mechanical shock wave within the balloon.

6. The catheter of claim 5, wherein the arc generator includes at least one electrode located within the balloon between the guide wire sheath and the outer wall of the balloon.

7. The catheter of claim 5, wherein the arc generator includes a pair of electrodes, each electrode of the pair of electrodes being located within the balloon between the guide wire sheath and the outer wall of the balloon.

8. A method comprising:
providing a catheter having an elongated carrier, a balloon about the carrier in sealed relation thereto and being arranged to receive a fluid therein that inflates the balloon, and a shock wave generator within the balloon;
inflating the balloon; and
causing the shock wave generator to form mechanical shock waves within the balloon from a point asymmetric within the balloon.

9. The method of claim 8, wherein the shock wave generator is an arc generator, and wherein the causing step includes providing the arc generator with voltage pulses.

10. The method of claim 8, wherein the catheter further includes a guide wire sheath having a guide wire lumen, and wherein the method further includes guiding the catheter along a guide wire received within the guide wire lumen to a desired position before inflating the balloon.
